(19) United States
(12) Patent Application Publication
(10) Pub. No.: US 2008/0187963 A1
(43) Pub. Date: Aug. 7, 2008

(54) Use of Lepa for Improving the Accuracy of Protein Synthesis in Vitro

(75) Inventors: Knud H. Nierhaus, Berlin (DE); Yan Qin, Berlin (DE); Daniel N. Wilson, Berlin (DE)

Correspondence Address: ROTHWELL, FIGG, ERNST & MANBECK, P.C., 1425 K STREET, N.W., SUITE 800, WASHINGTON, DC, 20005, US

(73) Assignee: Max-Planck Gesellschaft zur Foerderung der Wissenschaften E.V., Munchen (DE)

(21) Appl. No.: 11/994,211

(22) PCT Filed: Jul. 4, 2006
(86) PCT No.: PCT/EP06/06503

§ 371 Date: Dec. 28, 2007

(30) Foreign Application Priority Data
Jul. 4, 2005 (EP) 05 014 471.6

(51) Int. Cl. C12P 21/00; C07H 21/00

(52) U.S. Cl. 435/69.1; 536/23.1

(57) ABSTRACT

The present invention relates to methods, systems, compositions and kits for the synthesis of proteins in vitro, wherein the protein synthesis is carried out in the presence of the ribosomal factor LepA in order to significantly improve the accuracy of protein synthesis.

### DESCRIPTION

The present invention relates to methods, systems, compositions and kits for the synthesis of proteins in vitro, wherein the protein synthesis is carried out in the presence of the ribosomal factor LepA in order to significantly improve the accuracy of protein synthesis.

Systems for the in vitro synthesis of proteins are offered commercially and are important tools for structural and functional studies of proteins. Examples of the usage of these systems include the synthesis of toxic proteins that might be difficult to express in vivo, expression of heterologous proteins from organisms that might be difficult to cultivate in order to crystallize and/or to perform functional studies, synthesis of proteins doted with deuterium, ¹³C and ¹⁵N incorporation for NMR structure determination in solution, incorporation of artificial amino acids, such as selenomethionine, at specific protein positions for crystallization or pharmaceutical applications etc.

The most comprehensive and efficient in vitro systems for protein synthesis are coupled transcription/translation systems with bacterial cell lysates, where one adds, for example, T7 polymerase and a plasmid carrying a gene under a T7 promoter, e.g. Roche RTS 100 E. coli HY Kit, Roche RTS 500 E. coli HY Kit, Promega TNT Quick coupled Transcription/Translation Systems together with Promega E. coli T7 S30 Extract System for circular DNA, etc. The T7 transcript programs the translational apparatus of the lysate yielding up to 7 mg of synthesized protein per ml.

The major drawback of the currently available systems is the low accuracy with which the proteins are produced, i.e., the active fraction of distinct proteins can be as low as 30% of the total protein fraction for a given protein, therefore compromising the use of these protein products for subsequent molecular analysis. Surprisingly, it was found that addition of the ribosomal factor LepA improves the accuracy of the synthesized proteins to about 100% without significantly affecting the protein yield.

Thus, a first aspect of the invention relates to a method for synthesizing a protein in vitro by a translation system, particularly a bacterial translation system, wherein the protein synthesis is carried out in the presence of the ribosomal factor LepA.

A further aspect of the present invention relates to an in vitro translation system which comprises the ribosomal factor LepA.

Still a further aspect of the present invention relates to a reagent composition or kit for the in vitro synthesis of a protein comprising the ribosomal factor LepA.

Still a further aspect of the present invention is the use of the ribosomal factor LepA for increasing the accuracy of protein synthesis.

LepA was identified as a G-protein and is one of the most conserved proteins known in biology (Genebank Swiss-Prot: LepA from Escherichia coli: Entry name LEPA_ECO57; Primary accession number 60787; Genebank UniProt/TrEMBL: LepA orthologue from human: Entry name Q5XKM8_HUMAN; primary accession number Q5XKM8; protein name: FLJ13220). After EF-Tu (in archaea and eukarya EF1A) LepA is the second most conserved protein known with an amino-acid identity of 48 to 85% (Caldon et al., 2001). Sequence comparison suggested that it consists of five domains, the first four of which correspond to domains 1-3 and 5 of the elongation factor EF-G, respectively. In addition, LepA has a highly conserved C-terminal domain that has no sequence homology with any known proteins.

As shown in the example of the present application, LepA is a ribosomal factor which is capable of improving the accuracy of a protein synthesis particularly in an in vitro translation system.

The translation system can be any standard cell-free translation system, e.g, a bacterial system, which is supplemented by LepA. The system comprises (a) a translatable RNA encoding the protein to be synthesized and (b) a cell-free preparation comprising components of the cellular translation apparatus. Perferably the system is a coupled transcription/translation system. The transcription/ translation system preferably comprises (a1) a template nucleic acid encoding the protein to be synthesized operatively linked to an expression control sequence, (a2) a polymerase capable of producing translatable RNA from the nucleic acid (a1) and (b) from which the translatable RNA can be obtained by transcription. The system may be a prokaryotic or a eukaryotic system, preferably a prokaryotic system.

The system comprises translatable RNA encoding the protein to be synthesized and components of the cellular translation apparatus capable of translating the RNA. Preferably the system further comprises a template nucleic acid from which the translatable RNA can be obtained, e.g. by transcription or replication. In a preferred embodiment, the template nucleic acid is a DNA-molecule, e.g. a plasmid, encoding the protein to be synthesized operatively linked to an expression control sequence. The nucleic acid is expressed by a DNA-dependent RNA polymerase capable of transcribing the nucleic acid. On the other hand, the nucleic acid may be an RNA which may be replicated by an RNA-dependent RNA polymerase or replicase. The translatable RNA contains prokaryotic or eukaryotic translation signals, which are recognized by the components of the translation apparatus present in the system.

In a preferred embodiment, the expression control sequence is a heterologous promoter, such as a T7 or related promoter, e.g. a SP6 promoter, and the polymerase is a heterologous polymerase, such as a T7 RNA polymerase or related polymerase, e.g. SP6 RNA-polymerase.

Alternatively, the promoter may be a native cellular promoter and the polymerase is a native cellular DNA-dependent RNA polymerase.

The components of the cellular translation apparatus in the in vitro system are preferably provided by a translation-competent cell extract. The cell extract is preferably a cellular lysate, more preferably an extract or lysate from a prokaryotic cell, e.g. from E.coli cells or cells from another bacterial gram-negative prokaryotic cell or from a gram-positive prokaryotic cell, such as a B. subtilis cell.

In addition to the components as indicated above, the system may comprise usual components required for translation and optionally transcription or replication, such as ribonucleotides for RNA synthesis, amino acids for protein synthesis, a suitable biological energy source, such as ATP, acetylphosphate, phosphoenolpyruvate plus pyruvate kinase and similar systems.

The ribosomal factor LepA which is used to supplement the transcription/translation system, may be a prokaryotic or eukaryotic (e.g. mitochondrial) LepA, preferably a prokaryotic LepA, e.g. a LepA protein from E. coli. The LepA protein is preferably added as a homologous component to the system. It may be added as an isolated protein, e.g. purified from native or recombinant overproducing cells, or as a partially purified cell fraction. Further, the invention encompasses the use of functional LepA fragments or variants, e.g. LepA fragments or variants having ribosomal dependent GTPase activity still active in preventing errors. The invention further encompasses mutational altered elongation factor EF-G and fragments of EF-G that show LepA activity.

The amount of LepA in the system can be varied in a broad range in order to obtain a beneficial effect on the accuracy of protein synthesis without significantly reducing the efficiency of protein synthesis. For example, in a prokaryotic system, LepA is added in a molar ratio from about 0.05:1 to about 0.6:1, preferably from about 0.1:1 to about 0.5:1, most preferably from about 0.3:1 to about 0.4:1 to the amount of the 70S ribosomal subunit present in the system.

The methods, systems and reagent kits of the present invention are particularly suitable for the synthesis of proteins which are toxic in vivo, expression of proteins from organisms which are difficult to cultivate, or proteins which contain isotopes and/or artificial amino acids.

Furthermore, the present invention is to be explained in greater detail by the examples and figures hereinbelow.

### DESCRIPTION OF DRAWINGS

FIG. 1 Growth curves for E. coli cells of the strain BL21 under various conditions shown at the right side with the same color codes as those of the curves. The arrows indicate the addition of the inducer IPTG.

FIG. 2 Ribosome-dependent GTPase of EF-G (▪) and LepA (♦). The concentration of each factor was kept constant at 0.2 μM.

FIG. 3 Puromycin reaction of various ribosomal states. +, the peptidyl-residue of the P-tRNA is transferred to the puromycin at the A site of the peptidyl transferase center; −, no transfer occurs to puromycin.

FIG. 4 LepA induces a back-translocation (re-TL). The blue line is the reversed DNA transcript that indicates the ribosome position before translocation (third spot), the fourth spot a position three nucleotides shorter due to a translocation reaction. The last spot shows the back-translocation after adding LepA to the post-translocational state.

FIG. 5 A, synthesis of active GFP indicated by the fluorescent band in a native gel. B, the total synthesis derived from scanning the GFP band in an SDS gel is indicated with the blue line. The pink line indicates the amount derived from the fluorescent band of GFP in a native gel (A), the green band indicates the active fraction of the synthesized GFP. B, the active fraction of luciferase in the presence of increasing amounts of LepA, C, comparison of the LepA effect (pink, LepA:70S=0.3:1) on the active fraction of GFP (left) and luciferase (right).

### EXAMPLE

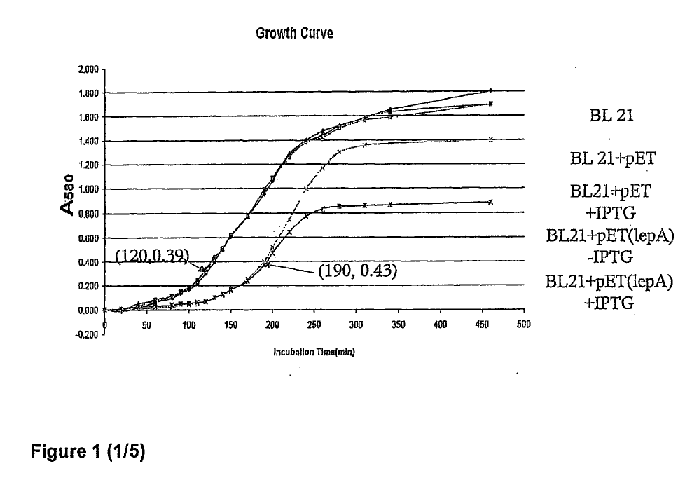

We fished the LepA gene from the E. coli genome and cloned it into a plasmid pET14b, which was under the control of a T7 promoter and added a His_{B}-tag to the N-terminus of the protein. First, we determined the effects of overexpression of LepA on the growth of E. coli cells BL21(DE3)physS according to manufacturers instruction (Novagen). FIG. 1 shows that even without induction of the LepA expression the growth only starts after a prolonged lag phase and enters earlier into the stationary phase compared with the control strains. This is expected since in the E. coli expression strain the T7 polymerase is under the control of a leaky LacZ promoter, thus allowing expression of the LepA from the plasmid without IPTG induction. The growth inhibition effects were much more severe after IPTG induction of LepA expression.

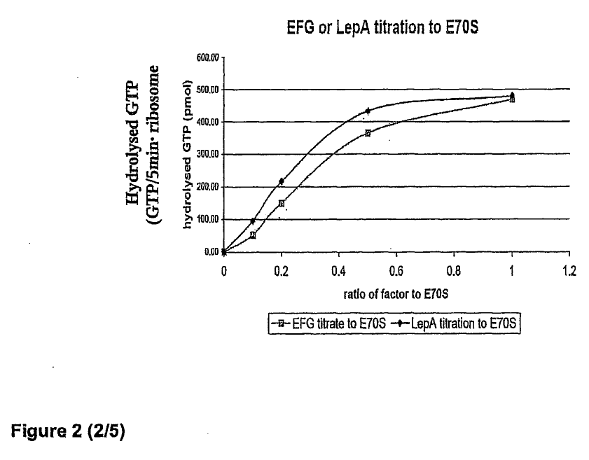

The growth stopped at a lower cell density, demonstrating that over-expression of the LepA is lethal to the cell. Next, the LepA protein was isolated after induction of expression and soluble protein purified via a Ni²⁺-column under native conditions and then tested in various functional assays. The first functional analysis was a test of a possible ribosome dependent GTPase activity of LepA according to {Dasmahapatra, 1981 #14727} with the buffer system described in {Dinos, 2004 #14684}. Since LepA might be an evolutionary offspring from the EF-G gene, we compared the LepA GTPase activity with that of EF-G, which is known to have one of the strongest ribosomal dependent GTPase activities. FIG. 2 shows that LepA not only has a ribosome dependent GTPase activity but that it is at least as strong as that of EF-G. With the exception of the indirect evidence of Mankin and co-workers that LepA cross-linked to oxazolidinones only when bound to the ribosome (see Colca et al., 2003), our data provide the first strong evidence that LepA is indeed a ribosomal factor. Control experiments indicate that LepA cannot translocate the tRNA₂·mRNA complex on the ribosome as EF-G.

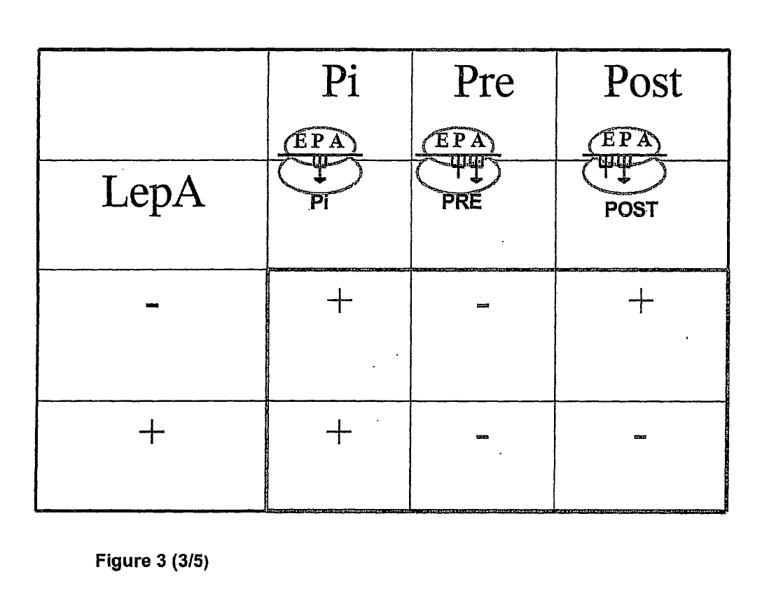

The next experiment was a surprise and gave the first hint of the function of LepA: When an analogue of a peptidyl tRNA was present at the P site and the adjacent E and A sites were free (a ribosome functional state referred to as the Pi state, i for initiation), LepA did not affect the puromycin reaction (according to {Bommer, 1996 #11801} in the buffer system described in {Dinos, 2004 #14684}), i.e. LepA did not prevent transfer of the aminoacyl moiety of the P site tRNA to the antibiotic puromycin that binds in the ribosomal A site of the peptidyltransferase centre. In contrast, in the post-translocational state LepA prevented a puromycin reaction (FIG. 3).

With the more laborious dipeptide analysis (see for example Marquez et al., 2004), this finding could be confirmed. The only possible explanation for these results was that LepA induces a so-called _{“}back-translocation” whereby the tRNAs are moved back from the P and E sites to the A and P sites and thus the puromycin reaction is prevented because the A site tRNA occupied the binding site of puromycin.

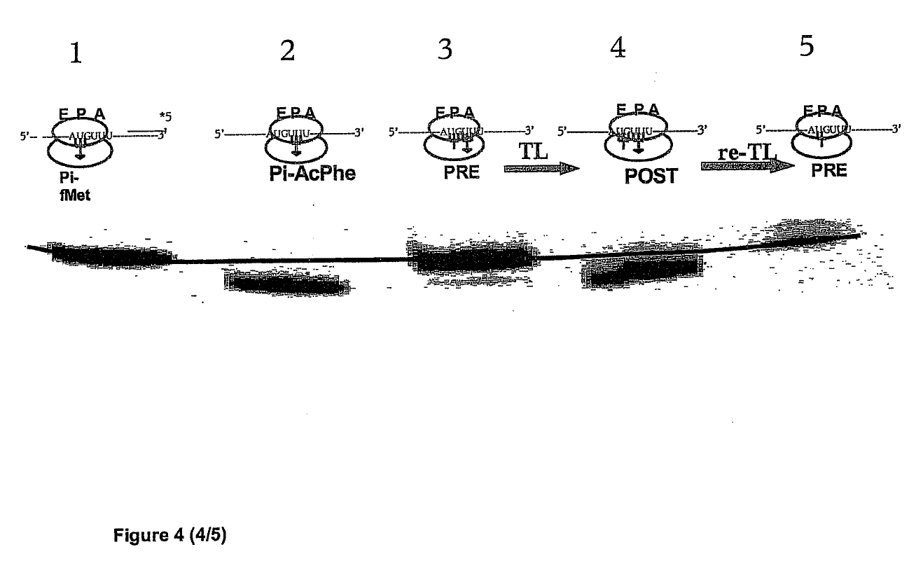

A direct test of this interpretation is a determination of the effect of LepA on the position of the mRNA relative to the ribosome using various ribosomal functional states. This method is called a _{“}footprinting assay” using reversed transcription as described in {Connell, 2002 #13483}. The assay measures the distance (via reverse-transcription) from a fixed point in the mRNA downstream of the ribosome (determined by a DNA primer complementary to a mRNA) to the ribosome. If the ribosome makes a translocation, the distance becomes shorter since the mRNA moves into the ribosome (in the 5′ direction), whereas if the ribosome makes a back-translocation the distance is increased. This is illustrated in FIG. 4. When LepA is added to a ribosome in a post-translocational state, the distance becomes longer (E). Therefore, LepA obviously has a unique function compared with all other known translocational factors since it induces a back-translocation.

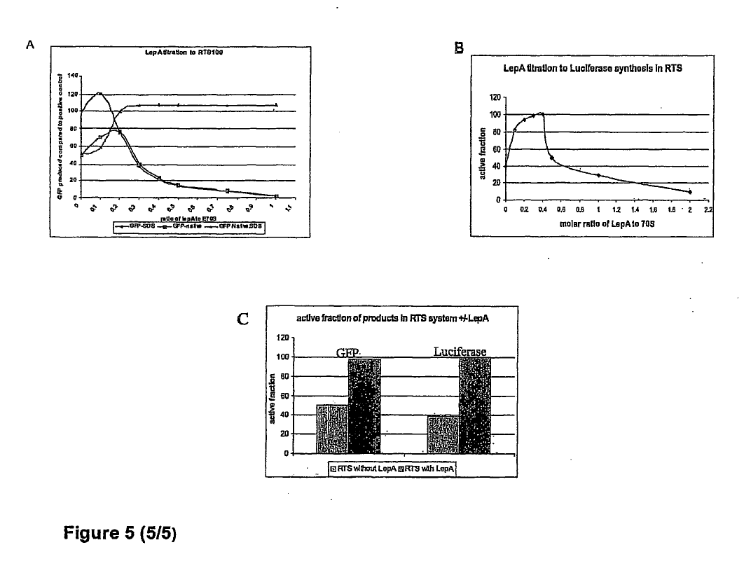

What is the function of this surprising back-translocation activity? FIG. 5B provides a possible answer. It shows the effect of LepA on the synthesis of GFP in a coupled transcription/translation system. At 0 of the x-axis, the 100% value (blue line) indicates the total synthesis derived from the GFP band intensity of an SDS gel electrophoresis. The pink spot again at the 0 position of the x-axis shows the active amount of synthesized GFP determined with a native gel electrophoresis shown in FIG. 5A (the active fraction was determined according to {Dinos, 2004 #14684}). In this experiment the active fraction of the synthesized GFP is about 50% in the absence of LepA (green line). Addition of LepA in a molar ratio to 70S of 0.2:1 shows a small reduction of the total yield for about 20%, but an increase of the active fraction to −100%. Further additions of LepA inhibit protein synthesis proportionally, eventually blocking it completely (>1 LepA per ribosome).

These in vitro results are consistent with the lethal effect observed in vivo (as shown in FIG. 1). What is particularly interesting/important is that all points during the decline of protein synthesis, the synthesized GFP is 100% active (green line in FIG. 5B).

### SUMMARY AND CONCLUSION

The results demonstrate that (i) the G-protein LepA is a ribosomal factor with a ribosome dependent GTPase that is at least as strong as EF-G. In spite of the structural relationship to EF-G it cannot translocate the tRNA₂·rmRNA complex. In fact, LepA induces the reverse reaction, namely a back-translocation that is probably related to the lack of the EF-G domain IV that might act as a _{“}door-stop” to prevent back-translocation. LepA heals the most important drawback of the current coupled translation systems, namely the inaccuracy of the current systems: The inactive fraction can be as large as 70% of the totally synthesized protein.

Addition of suitable amounts of LepA slightly reduces the total synthesis but increases the active fraction to virtually 100%. This is important if the structures of synthesized proteins should be determined via crystallization or after doting the synthesized protein with isotopes such as ¹³C or ¹⁵N for NMR. Likewise, an analysis of the function of the synthesized protein becomes prohibitively difficult by a large inactive fraction of the protein under observation. These drawbacks are overcome by the present invention.

### REFERENCES

Andersen, G. R., Nissen, P. And Nyborg, J. 2003. Elongation factors in protein biosynthesis. Trends Biochem, Sci. 28:434-441.
Butland, G., Peregrin-Alvarez, J. M., Li, J., Yang, W., Yang X., Canadien, V. Starostine, A., Richards, D., Beattie, B., Krogan, N., et al. 2005. Interaction network containing conserved and essential protein complexes in Escherichia col. Nature 433: 531-537.
Caldon, C. E., Yoong, P. and March, P. E. 2001. Evolution of a molecular switch: universal bacterial GTPases regulate ribosome function. Mol. Microbiol. 41: 289-297.
Colca, J. R., McDonald, W. G., Waldon, D. J., Thomasco, L. M., Gadwood, R. C., Lund, E. T., Cavey, G. S., Mathews, W. R., Adams, L. D., Cecil, E. T. et al. 2003. Crosslinking in the living cell locates the site of action of oxazolidinone antibiotics. J. Biol. Chem. 278:21972-21979.
Marquez, V., Wilson, D. N., Tate, W. P., Triana-Alonso, F. and Nierhaus, K. H. 2004. Maintaining the ribosomal reading frame: The influence of the E site during translational regulation of release factor 2. Cell 118:45-55.
Nierhaus, K. H. 1996. Protein synthesis—An elongation factor turn-on. Nature 379: 491-492.

1. A method for synthesizing a protein in vitro by a translation system, wherein protein synthesis is carried out in the presence of the ribosomal factor LepA.

2. The method according to claim 1, wherein the translation system comprises (a) a translatable RNA encoding the protein; and (b) a cell-free preparation comprising components of the cellular translation apparatus.

3. The method of claim 1, wherein the translation system is a prokaryotic system.

4. The method according to claim 1, wherein the cell-free preparation is a cell extract, particularly a cell lysate.

5. The method according to claim 4, wherein the cell extract is an extract from a prokaryotic cell, particularly an E.coli cell.

6. The method according to claim 1, wherein the system is a coupled transcription/translation system.

7. The method according to claim 6, wherein the transcription/translation system comprises (al) a nucleic acid encoding the protein to be synthesized operatively linked to an expression control sequence; (a2) a polymerase capable of producing translatable RNA from the nucleic acid and (b) a cell-free preparation comprising components of the cellular translation apparatus.

8. The method according to claim 7, wherein the expression control sequence is a heterologous promoter, such as a 17 or related promoter, and the polymerase is a heterologous polymerase, such as a 17 RNA polymerase or a related RNA polymerase, or wherein the expressions control sequence is a native cellular promoter and the polymerase is a native cellular DNA-dependent polymerase.

9. The method according to claim 1, wherein the synthesis is carried out in the presence of a prokaryotic LepA.

10. The method according to claim 9, wherein the LepA is from E. coli.

11. The method according to claim 1, wherein LepA is present in a molar ratio from about 0.05:1 to about 0.6:1 to the 70S ribosomal subunit present in the system.

12. An in vitro translation system which comprises added ribosomal factor LepA.

13. The system of claim 12 comprising (a) a translatable RNA encoding the protein to be synthesized operatively linked to an expression control sequence; (b) a cell-free preparation comprising components of the cellular translation apparatus, and (c) added ribosomal factor LepA.

14. The system of claim 12, which is a coupled transcription/translation system.

15. A reagent composition or kit for the in vitro synthesis of a protein comprising added ribosomal factor LepA.

16. Use of the ribosomal factor LepA for increasing the accuracy of protein synthesis.

17. The use of claim 16 in an in vitro system.

18. The use of claim 16 in an in vitro coupled transcription/translation system.
